(19) United States
(12) Patent Application Publication
(10) Pub. No.: US 2005/0163846 A1
(43) Pub. Date: Jul. 28, 2005

(54) Preparation composition containing acid-unstable physiologically active compound, and process for producing same

(75) Inventor: Shigeru Aoki, Gifu (JP)

Correspondence Address: NIXON & VANDERHYE, PC, 901 NORTH GLEBE ROAD, 11TH FLOOR, ARLINGTON, VA, 22203, US

(73) Assignee: EISAI CO., LTD., Tokyo (JP)

(21) Appl. No.: 10/849,544

(22) Filed: May 20, 2004

Related U.S. Application Data
(63) Continuation-in-part of application No. PCT/JP02/12132, filed on Nov. 20, 2002.

(30) Foreign Application Priority Data
Nov. 21, 2001 (JP) 2001-356018

(51) Int. Cl. A61K 9/24

(52) U.S. Cl. 424/472

(57) ABSTRACT

It is intended to disclose a preparation composition which enables convenient production of a sustained-release preparation composition which contains an acid-unstable physiologically active compound and which has the desired sustained-release property. In other words, the preparation composition according to the present invention comprises: a core containing an acid-unstable physiologically active compound; and a coating which covers the core and which contains a mixture of a water-insoluble polymer and an enteric polymer. The preparation composition of the present invention makes it possible to maintain an effective concentration in blood of the physiologically active compound over a long period of time, thereby improving its therapeutic effect. Accordingly, the present invention provides the composition that remains stable over prolonged storage. Moreover, the preparation composition of the present invention can be also used as a pulsed-release preparation.

### CROSS-REFERENCE TO RELATED APPLICATION

This is a continuation-in-part application of the Application PCT/JP02/12132 which was filed on Nov. 20, 2002.

### FIELD OF THE INVENTION

The present invention relates to a sustained-release or pulsed-release preparation composition. More particularly, the present invention relates to a preparation composition that exhibits continuous sustained release, or pulsed release, of a gastric acid secretion inhibitor as an acid-unstable active ingredient, and that is very suited to biological use, and to a process for producing the composition.

### DESCRIPTION OF THE RELATED ART

A benzimidazole-based compound used as a physiologically active compound, or an alkali metal salt, alkaline earth metal salt, or other physiologically acceptable salt thereof, is a peptic ulcer therapeutic drug that has a powerful inhibitory action on what are known as proton pumps, and that suppresses gastric acid very well. It is well known that examples of such therapeutic drugs at present include omeprazole, lansoprazole, rabeprazole, pantoprazole and the like.

The action of the above-mentioned compounds is continuous and more powerful than that of histamine H₂ receptor antagonists, and these drugs are currently administered just once a day. However, there have been early clinical indications that if these drugs are taken in the morning, it may be difficult to suppress the secretion of gastric acid during sleep.

Accordingly, it is predicted that therapeutic efficacy would be improved by imparting the desired sustained release property to a therapeutic agent containing the above-mentioned benzimidazole-based compound, and thereby maintaining the effective concentration of this agent in the blood.

Nevertheless, it is difficult to sustain release enough to maintain the blood concentration with a preparation composition in which the above-mentioned physiologically active compound (or a preparation composition containing this physiologically active compound) is covered only with an enteric polymer. Also, with a preparation composition in which the above-mentioned physiologically active compound (or a preparation composition containing this physiologically active compound) is covered only with a water-insoluble polymer, the dissolution of the physiologically active compound would be so slow that there is the possibility that this active compound will be decomposed in the gastric acid. Furthermore, if the coverage amount of water-insoluble polymer is increased in order to suppress decomposition in gastric acid, the dissolution of the active compound slows down to the point that a sufficient therapeutic efficacy will not be obtained.

It has been proposed that a benzimidazole-based compound (as the physiologically active compound) can be released gradually if a matrix is formed with a higher alcohol or a fatty acid ester (see Patent Document 1), but it is still possible that the physiologically active compound will be decomposed by gastric acid. It has also been proposed that a sustained-release preparation composition can be obtained by providing a dissolution suppressing film on the inside of an enteric coating of an enteric preparation containing omeprazole (see Patent Document 2).

However, it is known empirically that further covering the coating of a preparation already covered with the dissolution suppressing film can damage the dissolution suppressing film, thereby affecting the function thereof and also changing the dissolution behavior of the physiologically active compound in the preparation. Accordingly, considerable care must be taken in the coating step (operation) in which the dissolution suppressing film is covered. With a process based on this technology (see Patent Document 3), after the dissolution suppressing film has been applied, it is necessary to further cover the enteric coating on the film, so coating solutions for the film and for the coating have to be prepared separately, which causes a problem of complicating the production.

The present invention was conceived in light of these problems, and it is a particular object of the present invention to provide a preparation composition that enables the simple production of a sustained-release preparation composition containing an acid-unstable benzimidazole-based compound, that has the desired sustained release or pulsed release property, that maintains its effective blood concentration over an extended period of time and thereby improves therapeutic efficacy, and that is stable during extended storage, and to provide a process for producing the preparation composition.
Patent Document 1: WO 00/074654
Patent Document 2: WO 99/32091
Patent Document 3: WO 98/32091

### SUMMARY OF THE INVENTION

As a result of diligent investigation aimed at solving the above problems, the inventors completed the present invention upon discovering that an enteric coating having a dissolution control function can be produced by employing a coating that comprises a mixture of an enteric polymer and a water-insoluble polymer. Moreover, the inventors also discovered to their surprise that a preparation composition of the present invention not only has the desired sustained-release property, but also functions as a pulsed-release preparation when the pH value of a solution for dissolution is set to weak alkaline.

Specifically, the above object of the present invention is achieved by a preparation composition that comprises: (1) a core containing an acid-unstable physiologically active compound; and (2) a coating that covers the core, the coating comprising at least one layer and containing a water-insoluble polymer and an enteric polymer.

The coating of the above-mentioned preparation composition involves at least one layer and combines a dissolution control function with an enteric function, and also contributes to higher productivity.

Concerning a preferred aspect of the preparation composition according to the present invention, the water-insoluble polymer is contained in the amount of 20 to 80 wt % with respect to the total weight of the water-insoluble polymer and the enteric polymer which are contained in the coating.

Concerning a preferred aspect of the preparation composition according to the present invention, the coating further contains a plasticizer.

Concerning a preferred aspect of the preparation composition according to the present invention, the core further contains an alkaline substance.

Concerning a preferred aspect of the preparation composition according to the present invention, the water-insoluble polymer is selected from the group consisting of ethyl cellulose, aminoalkyl methacrylate copolymer, and shellac.

Concerning a preferred aspect of the preparation composition according to the present invention, the enteric polymer is selected from the group consisting of hydroxypropyl methyl cellulose acetate succinate, hydroxypropyl methyl cellulose phthalate, methacrylic acid-methyl methacrylate copolymer, and methacrylic acid-ethyl acrylate copolymer.

Concerning a preferred aspect of the preparation composition according to the present invention, the plasticizer is selected from the group consisting of triethyl citrate, cetyl alcohol, glycerol fatty acid ester, propylene glycol, polyethylene glycol, hydrogenated castor oil, hydrogenated rapeseed oil, and silicone oil.

Concerning a preferred aspect of the preparation composition according to the present invention, the alkaline substance is selected from the group consisting of sodium hydroxide, potassium hydroxide, sodium carbonate, and potassium carbonate.

Concerning a preferred aspect of the preparation composition according to the present invention, the physiologically active compound is a benzimidazole-based compound or a physiologically acceptable salt thereof.

Concerning a preferred aspect of the preparation composition according to the present invention, the benzimidazole-based compound is rabeprazole, and the physiologically acceptable salt thereof is a sodium salt.

Concerning a preferred aspect of this invention, the preparation composition according to the present invention is suitable for a sustained-release preparation.

Concerning a preferred aspect of this invention, the preparation composition according to the present invention is suitable for a pulsed-release preparation.

The above object is also achieved by a process for producing a preparation composition. The process comprises the step of spraying a solution containing a mixture of a water-insoluble polymer and an enteric polymer onto a core containing an acid-unstable physiologically active compound so as to form a coating that covers the core.

Concerning a preferred aspect of the process according to the present invention, the water-insoluble polymer is contained in an amount of 20 to 80 wt % with respect to the total weight of the water-insoluble polymer and the enteric polymer which are contained in the coating.

Concerning a preferred aspect of the process according to the present invention, the coating further contains a plasticizer.

Concerning a preferred aspect of the process according to the present invention, the core further contains an alkaline substance.

Concerning a preferred aspect of the process according to the present invention, the water-insoluble polymer is selected from the group consisting of ethyl cellulose, aminoalkyl methacrylate copolymer, and shellac.

Concerning a preferred aspect of the process according to the present invention, the enteric polymer is selected from the group consisting of hydroxypropyl methyl cellulose acetate succinate, hydroxypropyl methyl cellulose phthalate, methacrylic acid-methyl methacrylate copolymer, and methacrylic acid-ethyl acrylate copolymer.

Concerning a preferred aspect of the process according to the present invention, the plasticizer is selected from the group consisting of triethyl citrate, cetyl alcohol, glycerol fatty acid ester, propylene glycol, polyethylene glycol, hydrogenated castor oil, hydrogenated rapeseed oil, and silicone oil.

Concerning a preferred aspect of the process according to the present invention, the alkaline substance is selected from the group consisting of sodium hydroxide, potassium hydroxide, sodium carbonate, and potassium carbonate.

Concerning a preferred aspect of the process according to the present invention, the physiologically active compound is a benzimidazole-based compound or a physiologically acceptable salt thereof.

Concerning a preferred aspect of the process according to the present invention, the benzimidazole-based compound is rabeprazole, and the physiologically acceptable salt thereof is a sodium salt.

The term “acid-unstable” used in the present invention means the property that is chemically unstable in gastric acid and/or an aqueous solution with an acidic pH, and that promotes decomposition.

The term “core” used in the present invention refers to a core substance that only contains a physiologically active compound, or also contains various preparation additives normally used in the field of this technology, and refers to a substance in the form of a tablet, a granule, a fine granule, and the like.

Further, the term “preparation composition” used in the present invention means a preparation composition comprising a coated core, or a preparation composition in which the above-mentioned core is covered with an intermediate coating.

### BRIEF DESCRIPTION OF DRAWINGS

FIG. 1 is a graph showing the dissolution behavior of the preparation composition according to the present invention;

FIG. 2 is a graph showing the dissolution behavior of another preparation composition according to the present invention;

FIG. 3 is a graph showing the dissolution behavior of yet another preparation composition according to the present invention;

FIG. 4 is a graph showing the dissolution behavior of the preparation composition according to the present invention when a coverage amount was substantially constant (19.3±0.7 mg);

FIG. 5 is a graph showing the stability of the preparation composition according to the present invention in an acidic solution; and

FIG. 6 is a graph showing the results of the function of the preparation composition according to the present invention as a pulsed-release preparation.

### DESCRIPTION OF THE PREFERRED EMBODIMENTS

Preferred embodiments of the present invention will now be described in detail.

The present invention provides a preparation composition that comprises: a core containing an acid-unstable physiologically active compound; and a coating that covers this core, wherein the above-mentioned compound is unstable in acids, and the coating includes a mixture of a water-insoluble polymer and an enteric polymer. Moreover, the preparation composition according to this invention can be used not only as a sustained-release preparation, but also as a pulsed-release preparation by changing the pH value of a solution in which the preparation composition is dissolved.

The preparation composition according to the present invention is preferably a solid preparation composition, and can be formulated into any form that is ordinarily administered orally to humans, such as a tablet, a granule, a fine granule, a capsule and the like.

There are no particular restrictions on the acid-unstable physiologically active compound contained in the core used in the present invention, but examples of such compound include a gastric acid secretion inhibitor, an antibiotic and the like. Examples of the above-mentioned gastric acid secretion inhibitor include benzimidazole-based compound and a physiologically acceptable salt thereof, while examples of the above-mentioned antibiotic include penicillin-based antibiotics, macrolide-based antibiotics and the like.

Specific examples of the above-mentioned benzimidazole compound include rabeprazole, omeprazole, pantoprazole, lansoprazole and the like. Examples of physiologically acceptable salts thereof include alkali metal salt and alkaline earth metal salt. Specific preferred examples of alkali metal salt include sodium salt, potassium salt and the like, while preferred examples of the alkaline earth metal salt include magnesium salt and the like. The following are examples of the structural formulas of compounds suitable for use in the present invention.

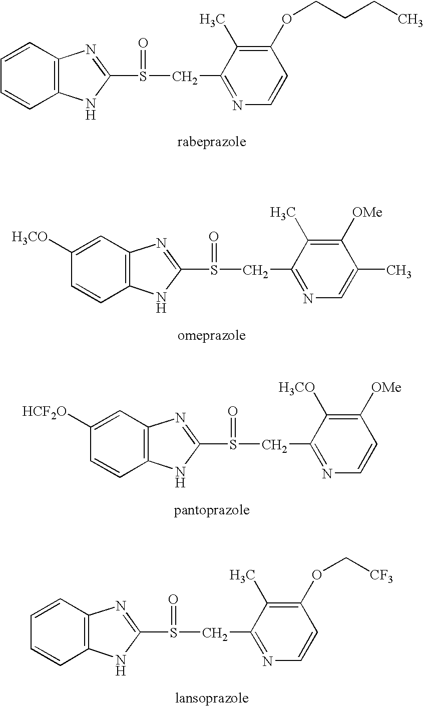

The benzimidazole-based compound used in the present invention can be produced by known methods. For instance, it can be produced by one of the methods disclosed in Japanese Laid-Open Patent Applications S52-62275, S54-141783, and H1-6270.

Concerning the present invention, it is particularly desirable that the benzimidazole-based compound be rabeprazole, and the physiologically acceptable salt thereof be a sodium salt.

A specific example of the above-mentioned penicillin-based antibiotics is benzyl penicillin, and specific examples of the above-mentioned macrolide-based antibiotics include erythromycin and clarithromycin.

The composition that forms the core used in the present invention (hereinafter referred to as “core composition”) is a composition that contains an acid-unstable physiologically active compound such as a benzimidazole-based compound, and that preferably contains any of various other preparation additives as well. There are no particular restrictions on the preparation additives, but examples of such additives include an excipient, a stabilizer, a disintegrant, a binder, a lubricant and the like.

The above-mentioned benzimidazole-based compounds are known to be extremely unstable in an acidic state. In core compositions and/or preparation compositions in which ordinary additives have been mixed, these compounds are extremely unstable when stored under conditions of elevated temperature and humidity, wherein decomposition is promoted. During this decomposition, not only is there an increase in impurities, but it has also been observed that core compositions and/or preparation composition containing a benzimidazole-based compound undergo a particularly pronounced color change.

An alkaline substance is preferably added to the core as a stabilizer. Specific examples of this alkaline substance include sodium hydroxide, potassium hydroxide, sodium carbonate, potassium carbonate and the like. These substances can be used singly or in combinations of two or more types.

The proportion in which the benzimidazole-based compound and the alkaline substance are mixed is preferably 0.001 to 2 parts by weight, and more preferably 0.005 to 1 parts by weight, and even more preferably 0.01 to 0.5 parts by weight of alkaline substance per 1 part by weight of benzimidazole-based compound.

The above method of stabilizing the benzimidazole-based compound with the alkaline substance according to-the present invention has a noticeably favorable effect in that it not only increases the stability of the benzimidazole-based compound itself, but also limits color changes.

Any excipient that is ordinarily used, such as lactose, mannitol and the like, can be used to produce the core containing the benzimidazole-based compound used in the present invention. There are no particular restrictions on the binder used in the present invention, but hydroxypropyl cellulose is preferable, and crospovidone is preferable as the disintegrant.

It is known that crospovidone, which is commonly used as the disintegrant, can be pulverized to reduce the disintegrating power and wetting power that are ordinarily associated with the disintegrant. Crospovidone that has been pulverized and/or sieved to a small particle size is used as the stabilizer of the benzimidazole-based compound in the present invention, and can be added in an amount over the amount (10% or less) in which it is usually added as the disintegrant. The average particle size of this pulverized and/or sieved crospovidone (hereinafter referred to as “fine crospovidone”) is preferably from a few microns to 50 μm.

Therefore, if crospovidone or fine crospovidone with an average particle size of from a few microns to 50 μm is used in the core composition or preparation composition according to the present invention, it will contribute, together with the above-mentioned alkaline substance, to better stabilization of the benzimidazole-based compound. Of course, fine crospovidone and ordinary crospovidone may also be used together.

The proportion in which the crospovidone and the benzimidazole-based compound are mixed in the present invention is preferably 0.1 to 10 parts by weight, and more preferably 0.2 to 8 parts by weight, and even more preferably 0.5 to 5 parts by weight of crospovidone per 1 part by weight of benzimidazole-based compound.

It should be noted that crospovidone frequently contains a trace amount of peroxide as an impurity, although the amount of peroxide varies with the manufacture and lot. Because the benzimidazole-based compound has the property of being readily oxidized, an antioxidant may be added at the time of the crospovidone addition. Specific examples of antioxidants include, but not limited to, sodium sulfite, sodium pyrosulfite, vitamin E, rongalite, thioglycerol, sodium thiosulfate, salt of ascorbic acid, acetylcysteine and the like.

The core according to the present invention can be produced by any commonly used method. For example, crospovidone, sodium hydroxide and/or potassium hydroxide, is mixed as a stabilizer into the benzimidazole-based compound, and the excipient or the binder is then added, thereby performing wet or dry granulation. If needed, crospovidone (used as the disintegrant) or the lubricant can be added and compression-molding can be performed to produce tablets. Of course, the process for producing the core composition used in the present invention is not limited to the above method.

Specific examples of the above-mentioned lubricant include magnesium stearate, sodium stearyl fumarate, calcium stearate, and stearic acid, as well as hydrogenated oils and other lipids. Among these examples, sodium stearyl fumarate, calcium stearate, and stearic acid are preferable, and sodium stearyl fumarate is particularly favorable.

There are no particular restrictions on the proportion in which the sodium stearyl fumarate or other lubricant is added in the present invention, but the amount is preferably 0.01 to 20 parts by weight and more preferably 0.05 to 15 parts by weight, and even more preferably 0.1 to 10 parts by weight, with respect to the uncovered core containing the benzimidazole-based compound or an alkali metal salt thereof which is the physiologically acceptable salt of the benzimidazole-based compound.

The above-mentioned lubricant preferably has an average particle size of 0.5 to 50 μm, and more preferably 1 to 50 μm.

The coating that covers the above-mentioned core will now be described. The coating according to the present invention is an enteric coating that contains a water-insoluble polymer and an enteric polymer. Because the enteric polymer generally exhibits acidity, direct contact of the enteric polymer with the benzimidazole-based compound, which is unstable to acids, is not desirable.

In view of this, it is preferable to provide an inert intermediate coating between the core containing the benzimidazole-based compound and the above-mentioned enteric coating for the preparation composition according to the present invention.

The term “inert” as used above means having no adverse effect on the stability of the benzimidazole-based compound.

There are no particular restrictions on the inert intermediate coating, which may be a coating containing a water-soluble polymer, a water-soluble or water-dispersible substance, or a water-insoluble substance, or may be a coating containing two or more types of these. Specific examples of the substance forming the intermediate coating include hydroxypropyl cellulose, hydroxypropyl methyl cellulose, aminoalkyl methacrylate copolymer E, lactose, mannitol, starch, crystalline cellulose, ethyl cellulose, vinyl acetate and the like.

As disclosed in Japanese Laid-Open Patent Application H1-29062, when the intermediate coating comprising a water-insoluble substance is provided, microparticles that are insoluble in water can also be mixed into the intermediate coating.

The coating according to the present invention is characterized by comprising at least one layer and containing a mixture of a water-insoluble polymer and an enteric polymer. The coating according to the present invention may also comprise two or more layers of coating with different mix ratios between the water-insoluble polymer and the enteric polymer, or may comprise a gradation coating in which the mix ratio between the water-insoluble polymer and the enteric polymer varies.

The desired sustained release property can be imparted with the present invention by suitably selecting the types of water-insoluble polymer and enteric polymer, the blend ratio of these polymers, and other such variables. Imparting this sustained release property makes it possible to control the rate of dissolution of the physiologically active compound that is the active ingredient.

There are no particular restrictions on the water-insoluble polymer used in the present invention as long as it substantially does not dissolve in water and has the property of dissolving or uniformly dispersing in methanol, ethanol, propanol, isopropanol, acetone, or other such organic solvents, but specific examples of the water-insoluble polymer include ethyl cellulose, aminoalkyl methacrylate copolymer, and shellac. Concerning the present invention, these substances can be used singly or in combinations of two or more types.

There are no particular restrictions on the enteric polymer used in the present invention, but specific examples of the enteric polymer include hydroxypropyl methyl cellulose phthalate (trade names HP-55 and HP-60, made by Shin-Etsu Chemical Industries), methacrylic acid-methyl methacrylate copolymer (trade names Eudragit L100, Eudragit L100-55, and Eudragit S100, made by Roehm Pharma), methacrylic acid-ethyl acrylate copolymer (trade name Eudragit L30D-55, made by Roehm Pharma), hydroxypropyl acetate succinate (Acoat, made by Shin-Etsu Chemical Industries) and the like. These substances can be used singly or in combinations of two or more types. Of these examples, it is preferable to use a methacrylic acid-methyl methacrylate copolymer (trade name Eudragit L100 or L100-55, also known as methacrylic acid copolymer L), Eudragit S100 (also known as methacrylic acid copolymer S, made by Roehm Pharma), or a mixture of these polymers.

In a preferred aspect of the present invention, the blend ratio between the water-insoluble polymer and the enteric polymer can be suitably selected from a range which enables the control of dissolution of the active ingredient, and the blend ratio can be varied, depending on application purposes in consideration of factors such as the physiologically active compound, the sustained release type, and the release rate.

In particular, to maintain the effective blood concentration of the benzimidazole-based compound for a long time, it is preferable for the water-insoluble polymer to be added in an amount of 15 to 85 wt %, and more preferably 20 to 80 wt %, and even more preferably 25 to 75 wt %, with respect to the total weight of the water-insoluble polymer and the enteric polymer in the composition that forms the coating. It will be difficult to maintain the effective blood concentration over an extended period of time if the water-insoluble polymer content is less than 20 wt %.

Various additives may be added to the coating which contains a mixture of the above-mentioned polymers and which is used to cover the core according to the present invention. Examples of such additives include a plasticizer, a colorant, and a lubricant.

Specific examples of the plasticizer used in the present invention include triethyl citrate, cetyl alcohol, glycerol fatty acid ester, propylene glycol, polyethylene glycol, hydrogenated castor oil, hydrogenated rapeseed oil, silicone oil and the like.

Specific examples of the colorant used in the present invention include a food dye, a lake dye, caramel, carotene, annatto (a natural dye derived from the annatto tree), cochineal, iron oxide, and titanium dioxide, as well as an opaque colorant whose main components are a lake dye and syrup. More specific examples include food aluminum lake dye, such as a food red No. 2 and No. 3, yellow No. 4 and No. 5, green No. 3, blue No. 1 and No. 2, violet No. 1, carmine (carminic acid aluminum salt), and pearl essence (whose main component is guanine), and the like.

Specific examples of the lubricant used in the present invention include magnesium stearate, talc, synthetic magnesium silicate, microparticulate silicon oxide and the like.

The amounts in which these additives are added, and when they are added, do not matter with the present invention as long as they are within the normal ranges employed in the field of pharmaceutical technology.

As to the method for covering the core according to the present invention with the coating containing the water-insoluble polymer and the enteric polymer, an enteric and sustained-release coating solution, which is prepared by dissolving or suspending the water-insoluble polymer and the enteric polymer, can be applied by means of, for example, pan coating, fluidized bed coating, or agitation fluidized bed coating. Specifically speaking, the following steps are taken: the core or a core that has been covered with an inert intermediate coating is agitated and/or fluidized to obtain a bed; the enteric, sustained-release coating solution produced by dissolving or suspending the water-insoluble polymer and the enteric polymer in a solvent is then sprayed onto the above-obtained bed; and the solvent is then removed by drying with hot forced air, thereby forming at least one layer of coating on the outer layer of the core.

A solution in which the water-insoluble polymer and the enteric polymer have already been adjusted to a specific blend ratio may be applied for the coating purpose, or a solution of the water-insoluble polymer and a solution of the enteric polymer may be prepared separately to form a gradation coating by varying the mix ratio of the two solutions over time with a pump or the like. It is also possible to laminate numerous layers of coatings with the same or different blend ratios of the water-insoluble polymer and the enteric polymer.

There are no particular restrictions on the solvent used for the above-mentioned coating solution as long as the water-insoluble polymer and the enteric polymer can both be dissolved or suspended therein, but examples of the solvent include: alcohols, such as methanol, ethanol, n-propanol, isopropanol, n-butanol, 2-methoxyethanol, and 2-ethoxyethanol; hydrocarbons, such as hexane, cyclohexane, petroleum ether, petroleum benzine, ligroin, benzene, toluene, and xylene; ketones, such as acetone and methyl ethyl ketone; halogenated hydrocarbons, such as dichloromethane, chloroform, carbon tetrachloride, ethylene dichloride, trichloroethylene, and 1,1,1-trichloroethane; esters, such as acetic acid methyl ester, acetic acid ethyl ester, and acetic acid butyl ester; and ethers, such as isopropyl ether and dioxane. These solvents should be selected according to the types of the water-insoluble polymer and the enteric polymer being used, and two or more types of the solvents can be combined as needed.

Of these examples, an alcohol is a more favorable solvent, and ethanol is even more preferable solvent.

As discussed above, the preparation composition according to the present invention comprises a core covered with a coating containing the water-insoluble polymer and the enteric polymer, but this preparation composition can be further covered with a moisture-proof coating. The term “moisture-proof coating” as herein used means a coating that suppresses the permeation of water vapor, and in terms of function, includes a coating in which the coating itself suppresses the permeation of water vapor, and a coating in which water vapor is trapped in the coating so that the water vapor cannot penetrate to the interior.

This moisture-proof coating has the function of improving stability by preventing the infiltration of moisture into the benzimidazole-based compound, and of preventing the cracking and deformation of the core composition that may be caused by swelling during the absorption of moisture by finely pulverized crospovidone.

This moisture-proof coating may be either water-soluble or water-insoluble, and examples of such moisture-proof coating include, but not limited to: coatings composed of polyvinyl acetal diethylaminoacetate, HA Sankyo (a mixture of fumaric acid, stearic acid, hydroxypropyl methyl cellulose, and polyvinyl acetal diethylaminoacetate), or polyvinyl alcohol; coatings produced by mixing one or more of cellulose, such as hydroxypropyl cellulose, hydroxypropyl methyl cellulose, and other cellulose derivatives; and/or sugar coatings whose main component is sucrose; and sugar coatings whose main component is maltitol.

The present invention will now be described in more specific terms through examples, but the present invention is not limited in any way by these examples. The following is the description of having a desired sustained release property of the preparation composition according to the present invention.

### EXAMPLE 1

Granulation was conducted by adding 6.4 kg of mannitol, 2.25 kg of crospovidone, and 0.2 kg of hydroxypropyl cellulose to and mixing these substances with 1.5 kg of a sodium salt of rabeprazole, and by adding the mixture to 3.75 kg of ethanol in which 0.1 kg of sodium hydroxide was dissolved. The granules thus produced were dried for 12 hours at 50° C., and then were caused to pass through a 1.5 mm screen. Subsequently, 0.6 kg of crospovidone and 0.15 kg of sodium stearyl fumarate (made by Penwest) were added to and mixed with the above-obtained granules, and the resultant mixture was tableted in a rotary compression machine. The tablets thus obtained each weighed 150 mg. Then, 3 kg of tablets were put into a coating pan and were coated with an intermediate coating of the following composition, in an amount of 5.7 mg per tablet.

| | |
|---|---|
| hydroxypropyl cellulose | 500 g |
| magnesium stearate | 125 g |
| ethanol | 10,000 g |

Next, 200 g of the tablets covered with the intermediate coating were used, and an enteric, sustained-release coating of the following composition, in an amount of 9.8 mg, was applied to the tablets, using a pan coating machine.

| | |
|---|---|
| Eudragit L100 | 40 g |
| ethyl cellulose | 40 g |
| talc | 7.5 g |
| titanium dioxide | 4.5 g |
| cetyl alcohol | 8 g |
| ethanol | 1250 g |

### EXAMPLE 2

Two hundred grams (200 g) of the tablets covered with the intermediate coating obtained in Example 1 were used, and the same enteric, sustained-release coating solution was sprayed onto the tablets, using a pan coating machine in the same manner as in Example 1, thereby applying a coating in an amount of 14.2 mg.

### EXAMPLE 3

Two hundred grams (200 g) of the tablets covered with the intermediate coating obtained in Example 1 were used, and the same enteric, sustained-release coating solution was sprayed onto the tablets, using a pan coating machine in the same manner as in Example 1, thereby applying a coating in an amount of 18.7 mg.

### EXAMPLE 4

Two hundred grams (200 g) of the tablets covered with the intermediate coating obtained in Example 1 were used, and the same enteric, sustained-release coating solution was sprayed onto the tablets, using a pan coating machine in the same manner as in Example 1, thereby applying a coating in an amount of 31.5 mg.

### EXAMPLE 5

Two hundred grams (200 g) of the tablets covered with the intermediate coating obtained in Example 1 were used, and the following enteric, sustained-release coating solution was sprayed onto the tablets, using the same pan coating machine as in Example 1, thereby applying a coating in an amount of 8.5 mg.

| | |
|---|---|
| Eudragit L100 | 60 g |
| ethyl cellulose | 20 g |
| talc | 7.5 g |
| titanium dioxide | 4.5 g |
| cetyl alcohol | 8 g |
| ethanol | 1250 g |

### EXAMPLE 6

Two hundred grams (200 g) of the tablets covered with an intermediate coating obtained in Example 1 were used, and the same enteric, sustained-release coating solution as in Example 5 was sprayed onto the tables, using a pan coating machine in the same manner as in Example 1, thereby applying a coating in an amount of 13.7 mg.

### EXAMPLE 7

Two hundred grams (200 g) of the tablets covered with an intermediate coating obtained in Example 1 were used, and the same enteric, sustained-release coating solution as in Example 5 was sprayed onto the tables, using a pan coating machine in the same manner as in Example 1, thereby applying a coating in an amount of 19.2 mg.

### EXAMPLE 8

Two hundred grams (200 g) of the tablets covered with an intermediate coating obtained in Example 1 were used, and the same enteric, sustained-release coating solution as in Example 5 was sprayed onto the tables, using a pan coating machine in the same manner as in Example 1, thereby applying a coating in an amount of 25.1 mg.

### EXAMPLE 9

Two hundred grams (200 g) of the tablets covered with an intermediate coating obtained in Example 1 were used, and the same enteric, sustained-release coating solution as in Example 5 was sprayed onto the tables, using a pan coating machine in the same manner as in Example 1, thereby applying a coating in an amount of 31.1 mg.

### EXAMPLE 10

Two hundred grams (200 g) of the tablets covered with the intermediate coating obtained in Example 1 were used, and the following enteric, sustained-release coating solution was sprayed onto the tablets, using a pan coating machine in the same manner as in Example 1, thereby applying a coating in an amount of 4.6 mg.

| | |
|---|---|
| Eudragit L100 | 20 g |
| ethyl cellulose | 60 g |
| talc | 7.5 g |
| titanium dioxide | 4.5 g |
| cetyl alcohol | 8 g |
| ethanol | 1250 g |

### EXAMPLE 11

Two hundred grams (200 g) of the tablets covered with the intermediate coating obtained in Example 1 were used, and the same enteric, sustained-release coating solution as in Example 10 was sprayed onto the tablets, using a pan coating machine in the same manner as in Example 1, thereby applying a coating in an amount of 9.4 mg.

### EXAMPLE 12

Two hundred grams (200 g) of the tablets covered with the intermediate coating obtained in Example 1 were used, and the same enteric, sustained-release coating solution as in Example 10 was sprayed onto the tablets, using a pan coating machine in the same manner as in Example 1, thereby applying a coating in an amount of 14.8 mg.

### EXAMPLE 13

Two hundred grams (200 g) of the tablets covered with the intermediate coating obtained in Example 1 were used, and the same enteric, sustained-release coating solution as in Example 10 was sprayed onto the tablets, using a pan coating machine in the same manner as in Example 1, thereby applying a coating in an amount of 20.0 mg.

### EXAMPLE 14

Two hundred grams (200 g) of the tablets covered with the intermediate coating obtained in Example 1 were used, and the same enteric, sustained-release coating solution as in Example 10 was sprayed onto the tablets, using a pan coating machine in the same manner as in Example 1, thereby applying a coating in an amount of 27.7 mg.

Control 1

Two hundred grams (200 g) of the tablets covered with the intermediate coating obtained in Example 1 were used, and the following enteric coating solution was sprayed onto the tables, using a pan coating machine in the same manner as in Example 1, thereby applying a coating in an amount of 20.0 mg.

| | |
|---|---|
| Eudragit L100 | 80 g |
| Talc | 7.5 g |
| titanium dioxide | 4.5 g |
| cetyl alcohol | 8 g |
| ethanol | 1250 g |

The tablets (preparation compositions) obtained in Examples 1 to 14 and Control 1 were evaluated by the following methods.

Evaluation Method 1

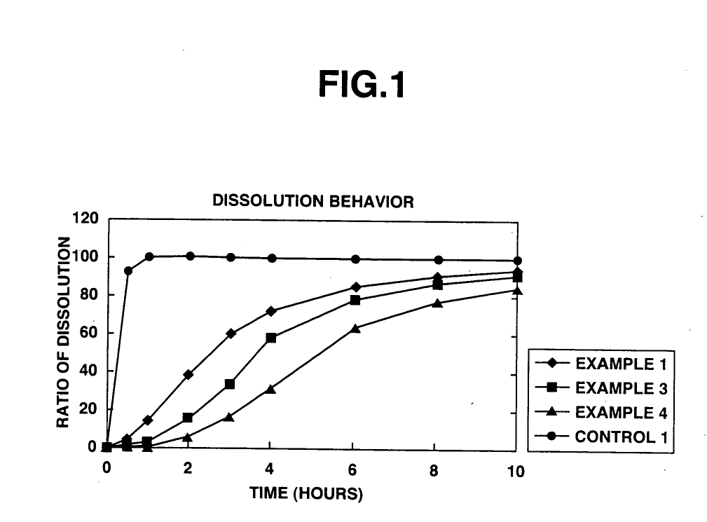

The tablets obtained in Examples 1, 3 to 7, and 9 to 14, and Control 1 were subjected to a dissolution test by paddle method in 1000 ml of a 0.1 M Na₂CO₃ aqueous solution (solution temperature: 37° C.) that had been adjusted to a pH of 11. The ratio of dissolution was measured by measuring the rabeprazole absorbance of the sampled solution by UV method. These results are given in FIGS. 1 to 4.

It can be seen from FIGS. 1 to 3 that dissolution occurred quickly with Contorol 1, which featured tablets covered with a Eudragit L100 coating (an enteric polymer) that did not contain a water-insoluble polymer. On the other hand, it was found that the rabeprazole, which is the physiologically active compound, eluted gradually in the various Examples.

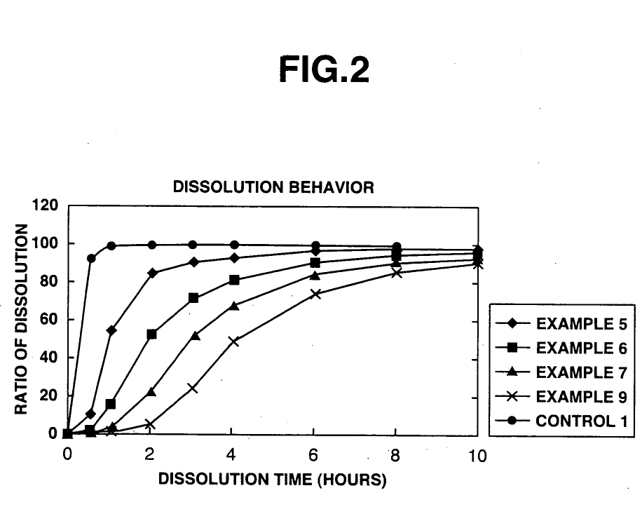

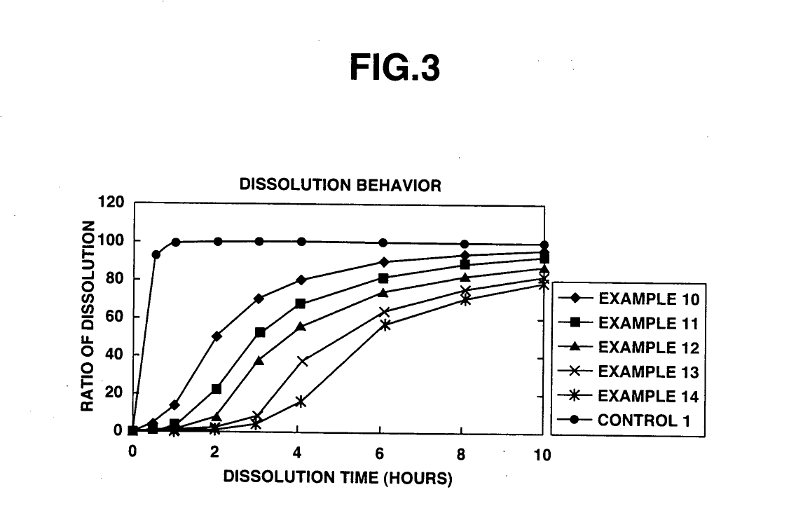

In Examples 1, 3, and 4 shown in FIG. 1, the compositions of the coatings are the same, but the coverage amounts of the coatings are different. Therefore, the dissolution ratio varies with the coverage amount. Specifically speaking, dissolution slows down as the coverage amount increases. Accordingly, and it was confirmed that the dissolution ratio can be controlled by varying the coverage amount of the coating. Similar results were obtained for FIGS. 2 and 3 as well.

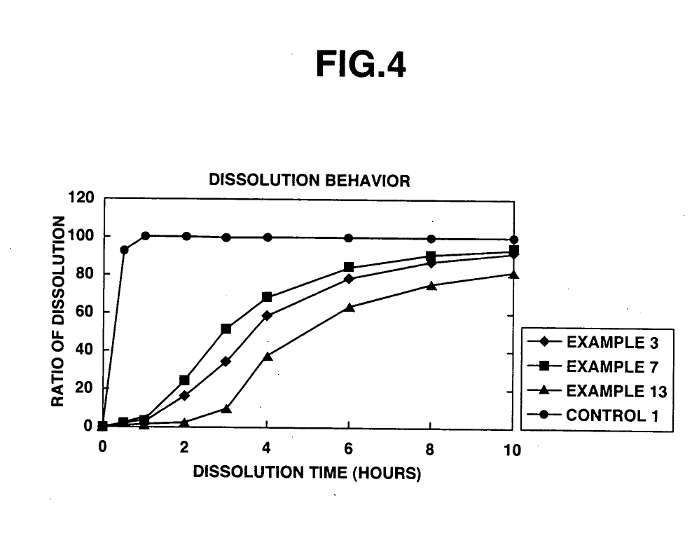

FIG. 4 is a graph showing the dissolution behavior of the preparation compositions prepared in Examples 3, 7, and 13 and Control 1 when the coverage amount of the coating according to the present invention was 19.3±0.7 mg.

As is clear from FIG. 4, when the coverage amount is substantially the same, the higher is the proportion in which the water-insoluble polymer is contained versus the total weight of the water-insoluble polymer and the enteric polymer in the coating, the more the dissolution ratio decreases. This proves that the dissolution ratio can be controlled by varying the blend proportions of the two polymers.

Evaluation Method 2

The stability of the tablets obtained in the Examples and the Control in an acidic solution was evaluated as described below.

There are two ways to evaluate dissolution under acidic conditions: acid resistant weight increase and acid resistance. Concerning acid resistant weight increase, a tablet is weighed at the outset and left for 2 hours in a test solution (37° C. solution temperature) with a pH of 1.0, and then weighed again to measure the weight increase.

Concerning acid resistance, a tablet is left for 2 hours in a test solution (37° C. solution temperature) with a pH of 1.0, and then the rabeprazole (active ingredient) content is measured, and the percentage remaining versus the content in the tablet prior to immersion in the test solution is calculated.

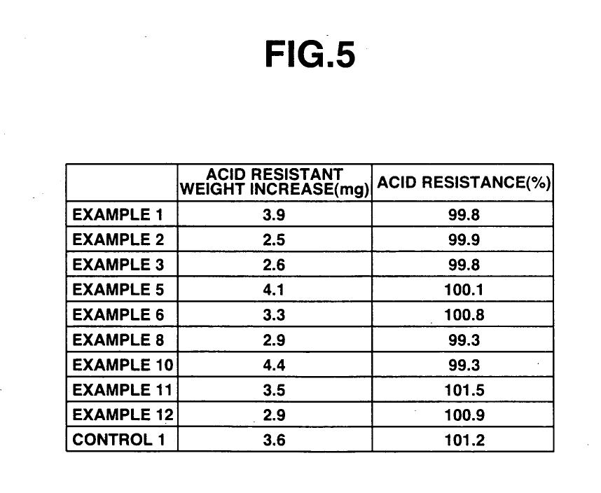

FIG. 5 shows the results for acid resistant weight increase and acid resistance in the various examples. The coating used in Control 1 was a conventional enteric coating, and a comparison of the results for this coating with those of the various Examples reveals that the rabeprazole was stable under acidic conditions in all of the Examples, and the coatings used in the various Examples had the function of an enteric coating.

The preparation composition according to the present invention can be used as a pulsed-release preparation as described below.

### EXAMPLE 15

Uncoated tables were produced in the following composition, and the uncoated tables were covered with an intermediate coating, and then with a sustained-release coating.

Granulation was conducted by adding 6.72 kg of mannitol, 2.4 kg of crospovidone, and 0.5 kg of hydroxypropyl cellulose to and mixing these substances with 1.0 kg of a sodium salt of rabeprazole, and by adding the mixture to 4 kg of ethanol in which 0.1 kg of sodium hydroxide was dissolved. The granules thus produced were dried for 20 hours at 50° C., and then were caused to pass through a 1.5 mm screen. Subsequently, 0.3 kg of crospovidone and 0.18 kg of sodium stearyl fumarate were added to and mixed with the granules, and the obtained mixture was tableted in a rotary compression machine. The (uncoated) tablets thus obtained each weighed 56 mg. Then, 3 kg of tablets were put into a coating pan, and an intermediate coating solution of the following composition was sprayed onto the tablets, thereby applying an intermediate coating in an amount of 3.7 mg per tablet. The intermediate coating solution was prepared by dissolving 318 g of ethyl cellulose and 540 g of hydroxypropyl cellulose in 16.0 kg of ethanol, and then by evenly dispersing 252 g of magnesium stearate in the above-obtained solution. Subsequently, a pan coating machine was used to apply a sustained-release coating (in the amounts of 5 mg, 10 mg, 20 mg, and 30 mg) of the following composition to the tablets covered with the intermediate coating (weight per tablet: 59.7 mg), thereby obtaining a sustained-release coating preparation that contains 5 mg of sodium rabeprazole. The sustained-release coating was prepared in the following composition:

| | |
|---|---|
| Eudragit L100 | 210 g |
| ethyl cellulose | 210 g |
| cetyl alcohol | 15 g |
| talc | 40 g |
| titanium dioxide | 25 g |
| ethanol | 6.25 kg |

Evaluation Method 3

The tablets obtained in Example 15 (the coverage amounts of the coating according to the present invention: 5 mg, 10 mg, 20 mg, and 30 mg) were dipped in 750 ml of a 0.1 N HCL,(hydrochloric acid) solution as an artificial gastric juice. Subsequently, 250 ml of 0.2 M Na₃PO₄ was added to the above solution, so that the pH value of the solution was adjusted to pH 6.8. The pH value of 6.8 corresponds to the pH value of an artificial intestinal juice (which is specified by the United States Pharmacopeia (USP) as a dissolution test solution for sustained-release preparations). The ratio of dissolution at each specified time was calculated by measuring, by the UV method, the rabeprazole absorbance (290 nm) of the solution sampled by using a flow cell.

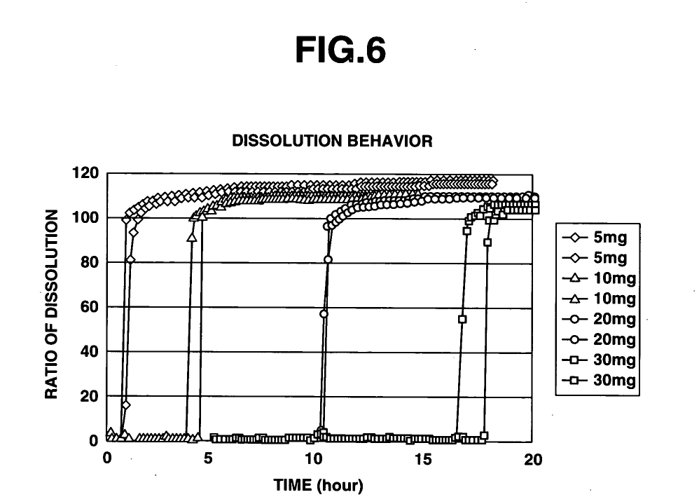

FIG. 6 is a graph showing the results of the dissolution behavior of the preparation composition prepared in Example 15 according to this invention. The time indicated by the horizontal axis of FIG. 6 is a period of time after the tablets were dipped in the artificial intestinal juice. The dissolution behavior shown in FIG. 6 was very different from the dissolution behaviors of the preparation compositions as the sustained-release preparations shown in FIGS. 1 to 4, and it was found that all the tablets of 5 mg, 10 mg, 20 mg, and 30 mg coating coverage amounts exhibited the behaviors as the pulsed-release preparations.

Referring to FIG. 6, the ratio of dissolution exceeds 100%. Because of the absorbance of rabeprazole decomposition product, the apparent value exceeding 100 % was observed.

### INDUSTRIAL APPLICABILITY

As described above, with the preparation composition according to the present invention, which is covered with a coating that is composed of a mixture of a water-insoluble polymer and an enteric polymer, and with the process for producing the same, it is possible to control the sustained release of an active ingredient covered with this coating by means of a simple process. Thus imparting the desired sustained release properties to an active ingredient affords an improvement in therapeutic efficacy in which the effective blood concentration in an organism is maintained over an extended period of time. Moreover, the preparation composition according to the present invention can be used not only as the sustained-release preparation, but also as the pulsed-release preparation by setting the pH value of a dissolving solution for the preparation composition to weak alkaline.

1. A preparation composition, comprising:
(1) a core containing an acid-unstable physiologically active compound; and
(2) a coating that covers the core, the coating comprising at least one layer and containing a water-insoluble polymer and an enteric polymer.

2. The preparation composition according to claim 1, wherein the water-insoluble polymer is contained in an amount of 20 to 80 wt % with respect to the total weight of the water-insoluble polymer and the enteric polymer which are contained in the coating.

3. The preparation composition according to claim 1, wherein the coating further comprises a plasticizer.

4. The preparation composition according to claim 1, wherein the core further comprises an alkaline substance.

5. The preparation composition according to claim 1, wherein the water-insoluble polymer is selected from the group consisting of ethyl cellulose, aminoalkyl methacrylate copolymer, and shellac.

6. The preparation composition according to claim 1, wherein the enteric polymer is selected from the group consisting of hydroxypropyl methyl cellulose acetate succinate, hydroxypropyl methyl cellulose phthalate, methacrylic acid-methyl methacrylate copolymer, and methacrylic acid-ethyl acrylate copolymer.

7. The preparation composition according to claim 3, wherein the plasticizer is selected from the group consisting of triethyl citrate, cetyl alcohol, glycerol fatty acid ester, propylene glycol, polyethylene glycol, hydrogenated castor oil, hydrogenated rapeseed oil, and silicone oil.

8. The preparation composition according to claim 4, wherein the alkaline substance is selected from the group consisting of sodium hydroxide, potassium hydroxide, sodium carbonate, and potassium carbonate.

9. The preparation composition according to claim 1, wherein the physiologically active compound is a benzimidazole-based compound or a physiologically acceptable salt thereof.

10. The preparation composition according to claim 9, wherein the benzimidazole-based compound is rabeprazole, and the physiologically acceptable salt thereof is a sodium salt.

11. The preparation composition according to claim 1, wherein the preparation composition is suitable for a sustained-release preparation.

12. The preparation composition according to claim 1, wherein the preparation composition is suitable for a pulsed-release preparation.

13. A process for producing a preparation composition, comprising the step of spraying a solution containing a mixture of a water-insoluble polymer and an enteric polymer onto a core containing an acid-unstable physiologically active compound so as to form a coating that covers the core.

14. The process according to claim 13, wherein the water-insoluble polymer is contained in an amount of 20 to 80 wt % with respect to the total weight of the water-insoluble polymer and the enteric polymer which are contained in the coating.

15. The process according to claim 13, wherein the coating further comprises a plasticizer.

16. The process according to claim 13, wherein the core further comprises an alkaline substance.

17. The process according to claim 13, wherein the water-insoluble polymer is selected from the group consisting of ethyl cellulose, aminoalkyl methacrylate copolymer, and shellac.

18. The process according to claim 13, wherein the enteric polymer is selected from the group consisting of hydroxypropyl methyl cellulose acetate succinate, hydroxypropyl methyl cellulose phthalate, methacrylic acid-methyl methacrylate copolymer, and methacrylic acid-ethyl acrylate copolymer.

19. The process according to claim 15, wherein the plasticizer is selected from the group consisting of triethyl citrate, cetyl alcohol, glycerol fatty acid ester, propylene glycol, polyethylene glycol, hydrogenated castor oil, hydrogenated rapeseed oil, and silicone oil.

20. The process according to claim 16, wherein the alkaline substance is selected from the group consisting of sodium hydroxide, potassium hydroxide, sodium carbonate, and potassium carbonate.

21. The process according to claim 13, wherein the physiologically active compound is a benzimidazole-based compound or a physiologically acceptable salt thereof.

22. The process according to claim 21, wherein the benzimidazole-based compound is rabeprazole, and the physiologically acceptable salt thereof is a sodium salt.
